(19) United States
(12) Patent Application Publication
(10) Pub. No.: US 2014/0076883 A1
(43) Pub. Date: Mar. 20, 2014

(54) Apparatus and method for per-operative modification of medical device stiffness

(71) Applicants: Vladimir Brailovski, Montreal (CA); Yvan Petit, Saint-Mathieu de Beloeil (CA); Jean-Marc Mac-Thiong, Mont-Royal (CA); Mark Driscoll, Notre-Dame-de-l'Ile-Perrot (CA); Stefan Parent, Saint-Lambert (CA); Hubert Labelle, Laval (CA)

(72) Inventors: Vladimir Brailovski, Montreal (CA); Yvan Petit, Saint-Mathieu de Beloeil (CA); Jean-Marc Mac-Thiong, Mont-Royal (CA); Mark Driscoll, Notre-Dame-de-l'Ile-Perrot (CA); Stefan Parent, Saint-Lambert (CA); Hubert Labelle, Laval (CA)

(21) Appl. No.: 14/025,912

(22) Filed: Sep. 13, 2013

Related U.S. Application Data
(60) Provisional application No. 61/703,388, filed on Sep. 20, 2012.

(51) Int. Cl. A61B 17/70; H05B 1/02

(52) U.S. Cl. CPC A61B 17/7002; H05B 1/025
USPC 219/491; 606/279; 219/506

(57) ABSTRACT

An apparatus for per-operative modification of a medical device stiffness comprising a body having at least two ends, an element attached to first end of said body providing heat and mechanical treatment and adapted to abut and deform at least one portion of the medical device, a second end, opposite to said first end of said body, adapted to be grasped, a means of connecting to a power source located anywhere between said ends of said body, whereby the apparatus provides a means to enable per-operative adjustment to the stiffness of a medical device.

### PRIORITY STATEMENT UNDER 35 U.S.C §.119 (E) & 37 C.F.R. §.1.78

This non-provisional patent application claims priority based upon the prior U.S. provisional patent applications entitled “Apparatus and method for per-operative modification of medical device stiffness”, application No. 61/703,388 filed Sep. 20, 2012 in the names of Vladimir BRAILOVSKI, Yvan PETIT, Jean-Marc MAC-THIONG, Mark DRISCOLL, Stefan PARENT, and Hubert LABELLE.

### TECHNICAL FIELD OF THE INVENTION

The present invention relates to a medical device and, more specifically, to an apparatus and method allowing per-operative adjustments of the material properties of a medical device.

### BACKGROUND OF THE INVENTION

Medical devices made from shape memory alloy material can exhibit a differentiation in stiffness moving from relatively flexible to rigid resulting from adjustment of the transition temperature and yield stress across selective segments. Medical devices implanted inside mammalian bodies are often submitted to a variety of forces and deformations while providing or assisting in the treatment of the underlying condition. Medical devices submitted to such an array of dynamic forces may be subject to additional manufacturing steps with the purpose of making them more suitable to perform appropriately in such a diverse mechanical environment. In one method, medical devices may be formed from several materials exhibiting different stiffness and joined together to create a composite material. In another method, medical devices may be differentially dimensioned throughout its cross section to offer selective stiffness. More recently, another method uses medical devices made of a monolithic shape memory alloy (SMA) material that may be pre-conditioned to exhibit variable stiffness as a function of reversible changes to its metallurgical structure. Stiffness, for the purpose of this patent application, refers to an objects ability to resist deformation in response to an applied force, alternatively known as modulus of elasticity as a response to stress. Depending on the particular manufacturing method, SMAs may exhibit both relatively flexible and rigid properties at the temperature of a mammalian body. To impart such relative flexibility, SMA may selectively undergo thermal and/or mechanical treatments rendering it superelastic of otherwise enabling it to exhibit stress-induced transformation from austenitic to martensitic phases. This distinct characteristic provides SMA with superelastic properties. Further improvements are available to better tailor the stiffness of medical devices to be better suited for their mechanical environment.

The present invention provides at least a partial solution to the identified problem.

### SUMMARY OF THE INVENTION

The aforementioned methods are known in the art of medical devices and offer surgeons the option of selecting a medical device exhibiting variable stiffness based on insight gained during pre-operative planning. However, one may never know with certainty what will be encountered during surgery. That is, during the clinical procedure the surgeon may desire to locally increase the rigidity or to locally increase the flexibility of at least one portion of a medical device in relation to another portion of the device. This per-operative decision may derive from the surgeon changing his surgical planning or approach during the operation based on case specific attributes of the patient under consideration. These changes in decision or surgical technique may occur during any surgical procedure and are well known to occur during spinal procedures.

For example, spinal instrumentation uses rigid rods and bone anchors to stabilize and treat spinal ailments. Often, spinal rods span at least two vertebral bodies and are engaged to one end of a bone anchor having another end affixed to the vertebrae. At times, the surgeon may employ fusion, or artificially induced ossification, to help solidify the surgically secured spinal segments. If fusion is not achieved nor desired, such instrumentation procedures leave previously mobile vertebral segments under a pseudo-static configuration which may lead to post-operative problems such as device loosening, fracture, and/or degeneration. In an attempt to rectify this recognized problem, several dynamic stabilization spinal devices have been proposed. These devices are suggested to alleviate stress or force concentrations found in vertebral segments adjacent to those having undergone fusion. Moreover, it is suggested that such a dynamic fixation would help prevent the otherwise sheltered biomechanical environments expected from static fixation of the adjacent segments which may encourage degeneration. Thus, a benefit is found in having a spinal fixation system offering both relatively flexible and rigid properties. Readily available technologies may in theory offer flexible and rigid properties; however, to date surgeons are limited to medical devices that are manufactured, as described above, to provide variable stiffness. Therefore, these manufactured devices do not grant the surgeon the option to make case-specific per-operative adjustments to the stiffness of the device. Consequently, there has been very little adoption of these technologies as it is near impossible to treat case-specific surgical reactions with generically manufactured devices.

It is therefore appreciated that a need exists to provide a method and apparatus to achieve per-operative adjustments to the stiffness of a medical device.

SMA may undergo transformation from relatively rigid to flexible and may undergo transformation from relatively flexible to rigid stiffness.

A preferred manner in which SMA may be transformed from relatively rigid to flexible, or to a state enabling superelastic deformation, involves mechanical followed by thermal treatments. A mechanical treatment may be performed in the form of cold working while thermal treatment may be achieved via annealing. Annealing time and temperature should generally be performed until recovery and polygonization occur while the recrystallization temperature of the alloy is not reached.

A preferred manner in which SMA may be transformed from relatively flexible to rigid, or from a state enabling superelastic deformation to a stable either austenitic or martensitic state at body temperature, involves thermal followed by mechanical treatments.

SMA devices used in mammalian bodies are subject to very stable temperatures around a body temperature of about 37 degrees Celsius. This allows the transition temperatures of SMA medical devices to be pre-conditioned to provide the following states in response to a change in force or stress at around body temperature: a heat-stable martensitic state defining a relatively flexible behaviour governed by plastic deformation, a heat-stable austenitic state defining a relatively rigid stiffness; a stress-transformable state enabling a stress-induced martensitic transformation defining a superelastic deformation or relatively flexible stiffness; or a heat-transformable state in which the transition temperatures of SMA medical devices may to be pre-conditioned so that when heated until about body temperature it enables the transformation from martensitic to austenitic states, otherwise known as thermoelastic martensitic transformation, defining a “memorized” shape transformation. More specific details of SMA behavior may be found in K. Otsuka, C. M. Wayman, Shape Memory Materials Cambridge University Press, 1999-10-07—300 pages, the contents of which are hereby incorporated in its entirety by reference.

U.S. Pat. No. 6,196,839 discloses a thermoelectric device and method to create temporary martensitic state SMA. An orthodontic cooling means is provided to temporarily induce martensitic SMA to an archwire so that it may be shaped to attach to the brackets found on maloccluded teeth. When the archwire is heated back to body temperature it converts to its austenitic state and tends back to its original shape providing corrective forces aimed at realignment of the teeth, thus making use of the heat-transformable state of SMA.

In a similar manner, patent application 2007/0173800 proposes a device and a method of achieving spinal correction. It is disclosed that a stable deformed martensitic state SMA spinal device is adjusted to match the spinal deformity while, upon local controlled heat treatment or moving from a cooled state to the higher body temperature, the SMA transforms to a austenitic state SMA which configuration tends towards the shape of a corrected spine thus providing corrective forces, thus making use of the heat-transformable state of SMA.

Patent applications 2007/0088412 and 2009/0218321 disclose a system for heating a SMA surgical device in a controlled manner. This is designed to shape transform the SMA via heat treatment regulated with a control system, thus making use of the heat-transformable state of SMA.

Patent application 2009/0107977 discloses a device to heat SMA in a controlled manner by passing current through the device to work the device into a target shape by generated heat, thus making use of the heat-transformable state of SMA.

Patent application 2006/0247679 discloses an apparatus and method for compressing a SMA implant using opposing dies attached to an actuator. This application seeks to temporally compress and reduce the volume of a SMA device, preferably under cool temperatures, therefore affording a surgical introduction of the device via a less invasive procedure. Compression is limited to about 8% strain to avoid cold working, alteration of the material's lattice, or over yielding. Upon reheating of the device following surgical insertion into a body the device retrieves its “memorized” shape, thus making use of the heat-transformable state of SMA.

These methods seek to transform SMA from a heat-transformable martensitic state to a heat-stable austenitic state, thus moving from martensitic to austenitic states, using direct or indirect thermal treatments. This induces a change in the shape of the device via its “memorized” properties. This is fundamentally different from transforming SMA between a heat-stable austenitic state and stress-transformable or heat-transformable states via thermal and/or mechanical treatments per-operatively to alter the mechanical properties of at least one portion the device, without necessarily altering its shape during this process, as proposed in the invention disclosed herein.

Transforming SMA from a heat-transformable state to a heat-stable austenitic state as defined in the aforementioned prior-art may be achieved by raising the temperature of the device to magnitudes of 40-100 degrees Celsius. Alternatively, the medical device may be in a heat-stable austenitic state at about body temperature and it is temporarily cooled to magnitudes of 10-20 degrees Celsius to allow deformation under the martensitic state, which, when reheated to about body temperature, the device returns to its “memorized” shape. These induced temperatures which may therefore be considered sustainable and may not induce tissue necrosis from overheating or over cooling thus allowing such devices and methods to be feasible. Otherwise, heat transfer to adjacent tissues may be easily controlled. The per-operative transformation of SMA from a relatively rigid heat-stable austenitic state to a relatively flexible stress-transformable state and contrariwise, as disclosed herein, requires much more elevated temperatures among the magnitudes of 200 to 900 degrees Celsius and strain deformations among the magnitude of 10 to 50%.

Clearly, none of the prior-art describe the applicants' instant invention. Although means of selectively heating or cooling an implanted SMA medical device to induce shape transformation and the likes are known, the clinical need to provide surgeons the ability to perform per-operative adjustments to the stiffness of a medical device remains unmet. The instant invention, disclosed hereinafter, provides a method and apparatus allowing surgeons to safely and effectively alter the stiffness of a medical device in a timely manner per-operatively.

It is therefore the object of the invention to provide a method and apparatus enabling per-operative modification of medical device stiffness. Another object of the invention is to provide a method and apparatus to render a portion of a medical device, formed at least in part of SMA, relatively flexible per-operatively. A further object of the invention is to provide a method and apparatus to render a portion of a medical device, formed at least in part of SMA, relatively rigid per-operatively. A further object of the invention is to provide a method and apparatus to render a portion of a medical device, formed at least in part of SMA, relatively more flexible or more rigid per-operatively. A further object of the invention is to provide a method and apparatus to render at least a portion of a medical device, formed at least in part of SMA, anisotropic or relatively flexible in one plane of deformation and relatively rigid or less flexible in another plane of deformation.

Numerous further advantages attend this invention.

In accordance with one embodiment of the invention, the invention provides an apparatus enabling the per-operative adjustment of the stiffness of a medical device. The apparatus includes a means of applying thermal and mechanical treatments to a localized area on a medical device in order to modify the stiffness of a medical device. The apparatus therefore include a heating means and may be coupled with a heat sink and or heat shield to help focus the heat treatment and shelter the surrounding tissue if the treatment is performed in a mammalian body. The apparatus may therefore also include a mechanical compression means, which may be accomplished by any known method of straining a material such as using apposing dies configured to compress the medical device, for example. The apparatus may further grant the user the ability to substantially select the desired stiffness to which they wish to alter the local portion of the medical device which may be visually or audibly revealed.

In accordance with another embodiment of the invention, the invention provided is directed to a method of performing the per-operative adjustment to the stiffness of a medical device. The method includes the act of adjusting or modifying the stiffness of a medical device by rendering it relatively flexible, relatively rigid, relatively more flexible or more rigid upon a second or a plurality of subsequent treatment(s) or any combination thereof. The method uses the ability of SMA material to undergo phase changes by controlled thermal and mechanical treatments. The method provides a means to adjust the stiffness of a medical device based on patient specific tissue behavior or reaction which may only be learned during an operation. The method therefore provides steps of heating and straining treatments, performed in any order, of at least one local portion of a medical device made at least in part of SMA.

### BRIEF DESCRIPTION OF THE DRAWINGS

These objects, advantages, and features of the present invention will become readily apparent upon reading the following non-restrictive drawings and descriptions of specific embodiments thereof, given the way of example only with reference to the accompanying drawings:

FIG. 1 depicts an embodiment of the invention where a spinal rod 10 has an unaltered or pre-manufactured stiffness or relatively rigid and a portion has been rendered to exhibit a relatively flexible stiffness;

FIG. 2 depicts yet another embodiment of the invention where a spinal rod 10 has an unaltered or pre-manufactured stiffness or relatively rigid and a portion has been rendered to exhibit a relatively flexible stiffness and a second portion has been rendered further flexible;

FIG. 3 depicts another embodiment of the invention where a spinal rod undergoes thermal and mechanical treatments per-operatively to render a portion of the spinal rod relatively flexible or rigid;

FIG. 4 depicts yet another embodiment of the invention where a spinal rod undergoes thermal and mechanical treatments per-operatively to render a portion of the spinal rod relatively flexible via a handheld apparatus having a head sink and a heat shield;

FIG. 5 depicts another embodiment of the invention where a spinal rod undergoes thermal and mechanical treatments per-operatively to render a portion of the spinal rod relatively flexible; and

FIG. 6 depicts another embodiment of the invention where any medical device cross section may be selectively altered to exhibit a relatively flexible and rigid stiffness.

FIG. 7 is a flow chart of an exemplary method in accordance with the teachings of the present invention.

### DETAILED DESCRIPTION OF THE INVENTION

Exemplary embodiments will be described to demonstrate the use, principles, and function of the invention disclosed herein. These descriptions and illustrations are non-limiting exemplary embodiments and no limitation to the scope of the invention is thereby intended. Any alteration or modification to the device or alternative application of the invention principles are contemplated to normally occur by those with ordinary skill in the art to which the invention relates.

The embodiments may be described as a multiple of distinct operations to facilitate comprehension of the present invention; however, it is to be appreciated that no such order dependence is inferred.

It is to be understood that in the discussed embodiments, thermal treatment includes but is not limited any means or combination of heat transfer via conduction, convection, or radiation such as direct or indirect resistance heating, Joule heating, applying heat via an inert gas, using an induction coil, using a laser, being brazed, and/or using a heated liquid.

It is to be understood that in the discussed embodiments, mechanical treatment includes but is not limited to mechanically working the alloy by one or several methods such as cold working, drawing, swaging, stretching, bending, and/or pressing.

It is to be understood that the material of the medical device may be made fully, at least partially, and/or from any combination of all suitable materials. These include but are not limited to shape memory metal alloys such as copper, nickel, titanium, niobium, tantalum, zirconium, silver, gold, zinc, iron and cadmium-based, shape memory polymers or thermoset plastics. Alternatively, any material capable of undergoing a modification to the stiffness is contemplated as being suitable. Because of its well-known biocompatibility and fatigue resistance it is preferable to use Nickel-titanium alloy for the application described herein.

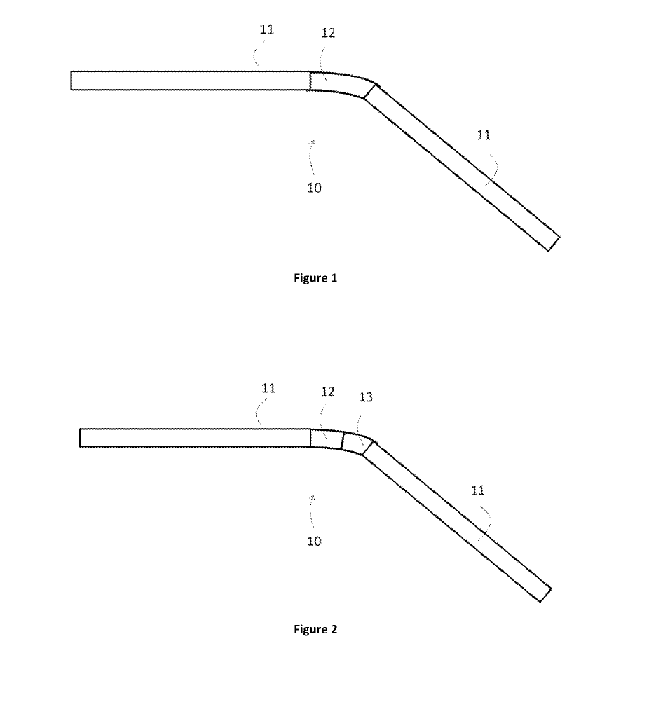

Referring now to the drawings in which like reference numerals indicate like parts, and in particular, to FIG. 1, one embodiment of the invention renders at least one portion of a medical device relatively flexible, exemplified herein as a spinal rod 10. During spinal surgery a patient's spine is exposed and instrumented using a combination of bone anchors and spinal rods. The bone anchors are affixed at one end to the spinal rod and theadingly engaged in the bone of the vertebral bodies at the other end. Before installation or once installed, it may be advantageous to modify the stiffness of certain portions of the spinal rod. Alternatively, it is understood that this invention is contemplated for any other medical device were a per-operative decision to alter the local stiffness of the device would be beneficial. The spinal rod 10 is being formed at least in part or in its entirety from a SMA having a portion exhibiting regular stiffness common to a generically pre-manufactured rod made of appropriate SMA material 11 and a portion which underwent heat and/or mechanical treatment rendering it relatively flexible 12. As implied, the relatively flexible portion may then be easily deformed, compared to the other non-treated portions of the rod or medical device, or may serve to grant the bone sites to which it is affixed a dynamic support. As exemplified in FIG. 2, if desired the surgeon may further render a portion of the device or spinal rod 10 more flexible or more rigid 13 by performing an additional thermal and/or mechanical treatment whereas the other portions of the device 11 remain unaltered.

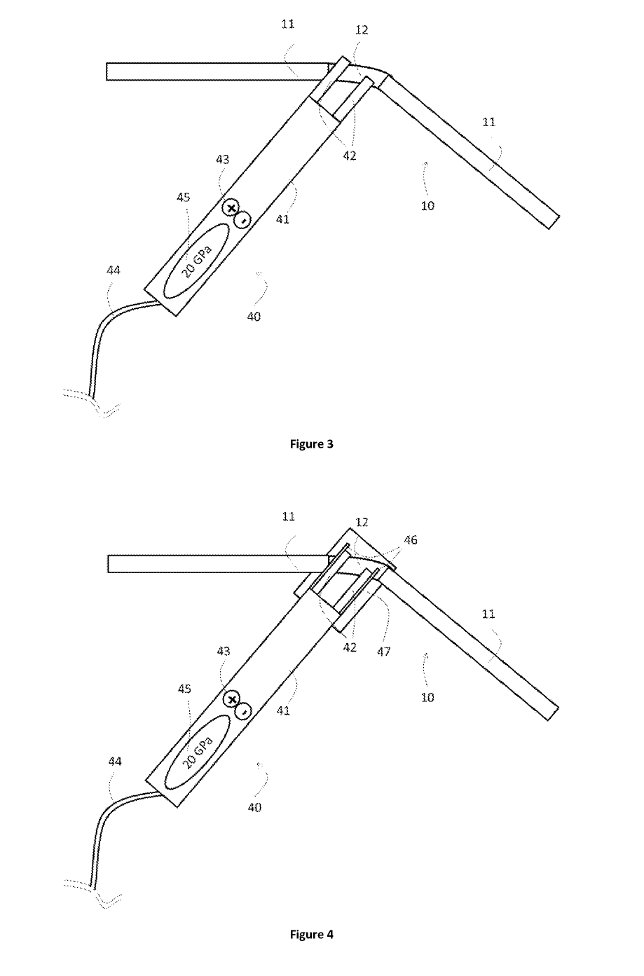

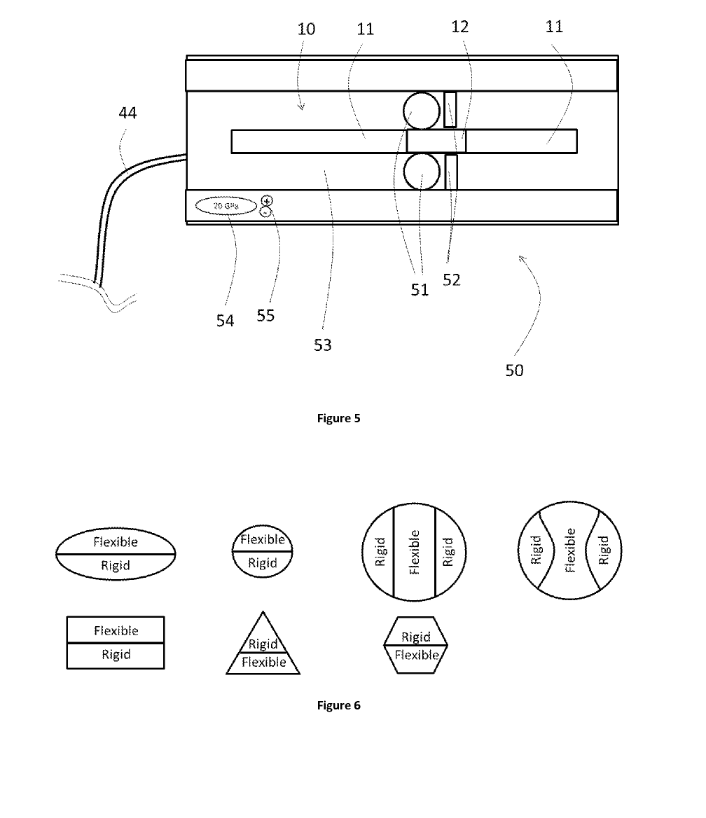

Referring now to FIGS. 3 and 4, an apparatus 40 is demonstrated for performing a per-operative adjustment to the stiffness of a medical device. One end of the apparatus 40, which is longitudinal in shape, is fitted elements 42 capable of providing sterile thermal and mechanical treatments of a medical device, shown here to be a spinal rod. As previously detailed thermal and mechanical treatments are contemplated to be possible in a number of different manners, the most preferable being direct resistance heating to temperatures in the range of 200-900 degrees Celsius for an approximate time of 5-20 minutes and straining in the range of 0-50%. The opposing end of the apparatus 40 is adapted to be grasped by a hand and includes a digital, analogue, or LED display 45 and perhaps coupled with an audible signal which may report the desired stiffness or modulus of the device and a means to control such a display 43. As a working example, if the surgeon would like a portion of the device to exhibit a stiffness of 20 GPa, the display control 43 is adjusted accordingly until the display 45 reflects this selection. Once selected, the apparatus 40 is engaged and then performs the appropriate thermal and mechanical treatments to the portion of the device to which such an adjustment is desired. Furthermore, positioned reasonably adjacent to the thermal and mechanical element 42, the apparatus 40 may also be fitted with a heat sink 46 and/or a heat shield 47. The heat sink and heat shield are designed to provide required heat sheltering to the surrounding tissues if the process is performed while the device or spinal rod 10 is inside a mammalian body while at least one end of the apparatus is sterile.

Referring now to FIG. 5, a table based apparatus is demonstrated for performing the per-operative adjustment to the stiffness of a medical device 50. Similarly to the hand held apparatus 40, the table based apparatus offers a means to alter the stiffness of a medical device except that this embodiment is performed per-operatively but while the medical device is out of the mammalian body being operated. The lower surface of the apparatus 50 is shape to rest securely on a substantially flat supporting surface. The upper portion of the apparatus 50 provides a surface to which the thermal and mechanical treatment means are affixed. In this particular embodiment the thermal 52 and mechanical 51 treatment elements are represented as being distinct but are also contemplated as being combined while effectively accomplishing their functions as previously disclosed for the handheld apparatus 40. The table based apparatus 50 also includes a display 54 and display control 55. In this embodiment, the surgeon may choose to render a selective portion of a medical device more flexible or rigid 12 while maintaining the generic pre-manufactures stiffness of the remaining portions of the device 11 during an operation, and may do so outside the mammalian body and conveniently in the operating room using this sterile table based apparatus 50. Using a number of appropriate identification methods, the surgeon may notch the location at which a modification to the stiffness is desired on the medical device, such as a spinal rod 10, and then do so aligning the notched location on the device 12 with the thermal and mechanical treatment elements housed on the table based apparatus 50.

It is to be understood that the apparatus 40 and 50 includes a power source of sorts capable supplying heating and mechanical power, such as a wire 44 being linked to an outlet providing electrical current for example. A means to convert AC current to DC current or a means to amplify the current may also be included in the apparatus. Moreover, heating of the device may otherwise be governed by a thermocouple or any other suitable temperature sensor. Such a temperature sensor serve to provide feedback to the apparatus' control system thus regulating the amount of current applied during direct resistance heating or any other heating means. It is also to be understood that any means of providing a mechanical advantage to the mechanical treatment element 42 of the apparatus 40 including as for example gears, levers, pneumatics, pulleys, etc. are contemplated herein and may be incorporated into the apparatus 40. A mechanical sensor is also provided to accurately control the degree a strain that is imposed on the medical device.

The hand held apparatus 40 and table based apparatus 50 may also serve as a tool to selectively render a medical device, or spinal rod 10, anisotropic. This is accomplished by performing a heat and mechanical treatment across a selected portion of the cross section of a medical device thus affording it a variable stiffness across its cross section. This is particularly beneficial if performed across a spinal rod as, at times, it may be desirable to have a relatively flexible stiffness in the bending and/or flexion of the sagittal plane granting it substantially dynamic fixation and a relatively rigid stiffness in the coronal plane granting it substantially static support. As demonstrated in FIG. 6, any appropriate cross section a medical device may adopt is contemplated and only a few are exemplified. To achieve such a control of the stiffness of the cross section, a heat sink will likely be required to maintain certain portions of the cross section at a lower temperature than the portion(s) undergoing a heat treatment while the mechanical treatment may be selectively introduced and controlled across the cross section. As it would be evident to one skilled in the art, any possible or beneficial combination or transition between relatively flexible and rigid stiffness is contemplated herein.

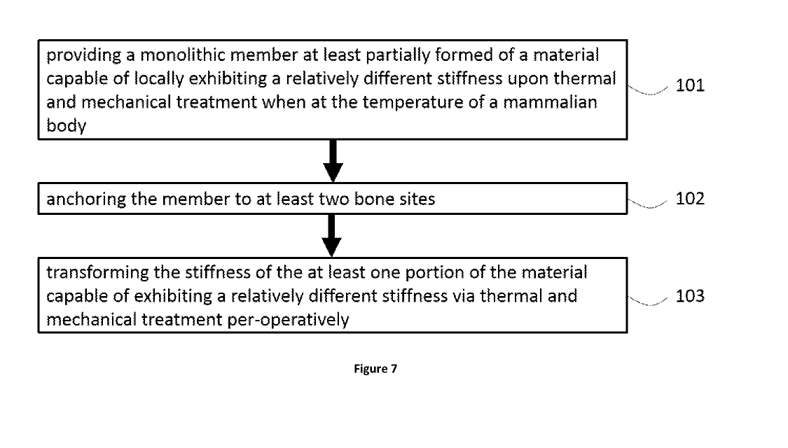

Referring now to FIG. 7, the methods associated with the use of the apparatus described herein first provide a monolithic member at least partially formed of a material capable of locally exhibiting a relatively different stiffness that the rest of the member upon thermal and/or mechanical treatment when at the temperature of a mammalian body 101. This member would then be anchored to at least two bone sites 102 and then transforming the stiffness of the at least one portion of the material capable of exhibiting a relatively different stiffness via thermal and mechanical treatment per=operatively.

In the course of the above described embodiments, a number of alternatives have been identified and others may well occur to those skilled in the art without departing from the field of the invention. Thus, various combinations, sub-combinations, and sundry adaptations are maintained under the principles of the provided invention.

Exemplary embodiments will be described to demonstrate the use, principles, and function of the invention disclosed herein. These descriptions and illustrations are non-limiting exemplary embodiments and no limitation to the scope of the invention is thereby intended. Any alteration or modification to the device or alternative application of the invention principles are contemplated to normally occur by those with ordinary skill in the art to which the invention relates.

The description of the present invention has been presented for purposes of illustration but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

1. An apparatus for per-operative modification of a medical device stiffness comprising:
a body having at least two ends;
an element attached to first end of said body providing heat and mechanical treatment and adapted to abut and deform at least one portion of the medical device;
a second end, opposite to said first end of said body, adapted to be grasped;
a means of connecting to a power source located anywhere between said ends of said body;
whereby the apparatus provides a means to enable per-operative adjustment to the stiffness of a medical device.

2. The apparatus in claim 1, wherein said body includes a display and controller as a means to view and set the desired stiffness of the medical devices.

3. The apparatus in claim 1, wherein said body includes a heat sink.

4. The apparatus in claim 1, wherein said body includes a heat shield.

5. The apparatus in claim 1, wherein said body includes a thermal sensor as a means to control the temperature of said one end.

6. The apparatus in claim 1, wherein said body includes a mechanical sensor as a means to control the amount of strain imposed of said one end.

7. The apparatus in claim 1, wherein said body, elements, and means of connecting a power source may be sterilized.

8. An apparatus for per-operative modification of a medical device stiffness comprising:
a body having at opposing surfaces;
a first surface adapted to be set on a level surface;
a second surface supporting at least a means of providing heat and mechanical treatments;
a heating element, supported by said second surface, providing a means of heat treatment;
a straining element, supported by said second surface and in relative proximity to said heating element, providing a means of mechanical treatment;
a means of connecting to a power source located anywhere on said body;
whereby the apparatus provides a means to enable per-operative adjustment to the stiffness of a medical device.

9. The apparatus in claim 8, wherein said body includes a display and controller as a means to view and set the desired stiffness of the medical devices.

10. The apparatus in claim 8, wherein said body includes a heat sink.

11. The apparatus in claim 8, wherein said body includes a heat shield.

12. The apparatus in claim 8, wherein said body includes a thermal sensor as a means to control the temperature of said heating element.

13. The apparatus in claim 8, wherein said body includes a mechanical sensor as a means to control the amount of strain imposed of said straining element.

14. The apparatus in claim 8, wherein said body, heating element, straining element, and means of connecting a power source may be sterilized.

15. A method for stabilizing at least two bone sites, comprising:
providing a monolithic member at least partially formed of a material capable of locally exhibiting a relatively different stiffness upon thermal and mechanical treatment when at the temperature of a mammalian body;
anchoring the member to at least two bone sites; and
transforming the stiffness of the at least one portion of the material capable of exhibiting a relatively different stiffness via thermal and mechanical treatment per-operatively.

16. The method of claim 15, wherein the monolithic member at least partially formed of a material capable of locally exhibiting a relatively different stiffness is mechanically pre-conditioned to exhibit relatively rigid properties and capable of locally exhibiting relatively flexible properties upon thermal treatment when at the temperature of a mammalian body.

17. The method of claim 15, wherein the monolithic member at least partially formed of a material capable of locally exhibiting a relatively different stiffness is thermally pre-conditioned to exhibit relatively flexible properties and capable of exhibiting relatively rigid properties upon mechanical strain treatment when at the temperature of a mammalian body

18. The method of claim 15, wherein the apparatus transforms a SMA from a heat stable austenitic state to a stress-transformable state.

19. The method of claim 15, wherein the apparatus further renders the material relatively more flexible or more rigid following a plurality of subsequent treatments.

20. The method of claim 15, wherein the medical device is rendered substantially anisotropic.
